(19) United States
(12) Patent Application Publication
(10) Pub. No.: US 2007/0166274 A1
(43) Pub. Date: Jul. 19, 2007

(54) 7-Dimethylamino-6-Demethyl-6-Deoxytetracycline Skin Treatment Kit

(76) Inventors: Leonard L. MAZUR, Mountain Lakes, NJ (US); Joseph J. Krivulka, (US)

Correspondence Address: PHARMACEUTICAL PATENT ATTORNEYS, LLC, 55 MADISON AVENUE, 4TH FLOOR, MORRISTOWN, NJ, 07960-7397, US

(21) Appl. No.: 11/459,778

(22) Filed: Jul. 25, 2006

Related U.S. Application Data
(63) Continuation-in-part of application No. 11/418,514, filed on May 4, 2006.
(60) Provisional application No. 60/760,121, filed on Jan. 19, 2006.

(51) Int. Cl. A61K 8/97; A61K 36/886; A61K 36/82

(52) U.S. Cl. 424/74; 424/744; 514/152; 424/729

(57) ABSTRACT

A method to ameliorate the skin-irritating effects of topical tretinoin treatment by providing the tretinoin-using patient with a skin-care kit which includes (1) topical tretinoin; and (2) a skin cleanser formulated to minimize tretinoin-induced skin irritation, and (3) a skin moisturizer formulated to reduce tretinoin-induced skin irritation; and (4) packaging to present the aforementioned components together as a unified system.

### RELATED APPLICATIONS

This application claims priority from provisional filing Ser. No. 60/760,121, filed 19 Jan. 2006, the contents of which are incorporated by reference. This application is a continuation in part of co-pending application Ser. No. 11/418,514, filed 4 May 2006, the contents of which are incorporated by reference.

### GOVERNMENT INTEREST

None.

### BACKGROUND

Acne vulgaris is a multifactorial skin disease that involves several processes:
Androgenic hormonal stimulation of the sebaceous glands, and abnormal desquamation of follicular keratinocytes in the pilosebaceous duct, leading to formation of microcomedones.
Excessive production of sebum.
Proliferation of P. acnes (Propionibacterium acnes) and follicular inflammation processes.
Production of inflammatory-inducing compounds (partially caused by the P. acnes population within the follicle), most notably neutrophil chemoattractants.
Changes in the permeability of the follicle wall, allowing release of bacterial antigens and inflammatory mediators, which drive the shift from non-inflammatory to inflammatory acne lesions.
   The literature suggests that a sound understanding of the pathophysiology of acne is key in determining optimal treatment. Therefore, appropriate, effective treatment will target:
normalization of follicular keratinization.
reduction of interfollicular P. acnes.
reduction of inflammation.
reduction of sebaceous gland activity.

Numerous topical medications are available for acne treatment, including retinoids and retinoid-like drugs, benzoyl peroxide, and antibiotics. Relatively less severe cases of acne can frequently be treated effectively with topical agents only. To avoid systemic toxicity, topicals are generally preferred to systemic therapy if favorable results can be maintained. In more severe cases of acne vulgaris, however, 7-Dimethylamino-6-demethyl-6-deoxytetracycline or an equivalent may be prescribed for oral administration, rather than topical administration.

We have invented a kit to treat acne vulgarism We expect this kit will improve patient compliance as compared to prior art approaches, and thus improve clinical outcomes. We expect our new kit will also decrease skin irritation and thereby increase the patient's satisfaction with both the acne vulgaris treatment itself, and with the prescribing physician.

Our solution involves providing the patient with kit which includes (1) a dermatologically-effective amount of 7-Dimethylamino-6-demethyl-6-deoxytetracycline, and (2) a skin cleanser formulated to minimize skin irritation, and (3) a skin moisturizer formulated to reduce skin irritation.

### BRIEF DESCRIPTION OF THE DRAWINGS

FIG. 1 illustrates an example of the claimed kit.

### DETAILED DESCRIPTION

Our invention is a kit which includes (1) a dermatologically-effective amount of 7-Dimethylamino-6-demethyl-6-deoxytetracycline, and (2) a skin cleanser formulated to minimize skin irritation, and (3) a skin moisturizer formulated to reduce skin irritation. We now discuss each component in turn.

### 7-Dimethylamino-6-demethyl-6-deoxytetracycline

7-Dimethylamino-6-demethyl-6-deoxytetracycline is a semi-synthetic derivative of tetracycline. It is chemically known as 4,7-Bis(dimethylamino)-1,4,4a,5,5a,6,11,12a-octahydro-3,10,12,12a-tetrahydroxy-1,11-dioxo-2-naphthacenecarboxamide. It is clinically called minocycline clinically. It has a structure as shown:

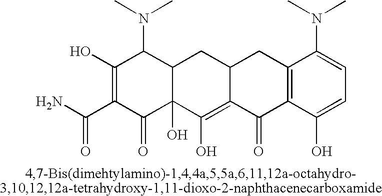

It may be synthesized by starting with tetracycline (either in free base or in a salt form, depending on whether solubility in polar or a non-polar solvent systems is desired). The reductive alkylation process may be accomplished by either chemical or catalytic reduction. One approach to doing this is taught by James H. BOOTHE et al., Reductive Alkylation Process, U.S. Pat. No. 3,148,212 (8 Sep. 1964). From this intermediate, the desired compound may be isolated by following the process described in Joseph PETISI et al., 7- and 9-Alkylamino-6-Deoxytetracycline, U.S. Pat. No. 3,226,436 (28 Dec. 1965).

The basis for the oral effectiveness of this compound includes its mechanism of action as an antibiotic. As a second generation tetracycline antibiotic, it acts as an anti-infective against the bacteria causing acne vulgarism It also reduces inflammation, as shown in clinical trials that have reported significant decreases in inflammatory lesions. It has a number of adverse side effects. These are outlined in Table 1:

TABLE 1

| | | | Reported Dose | | |
|---|---|---|---|---|---|
| | Test | | (Normalized | | |
| Organism | Type | Route | Dose) | Effect | Source |
| Infant | TDLo | oral | 12 mg/kg/2D-I | BRAIN AND | Therapie. Vol. 38, |
| | | | (12 mg/kg) | COVERINGS: | Pg. 93, 1983. |
| | | | | INCREASED | |
| | | | | INTRACRANIAL | |
| | | | | PRESSURE | |
| | | | | GASTROINTESTINAL: | |
| | | | | NAUSEA OR VOMITING | |
| Man | TDLo | oral | 343 mg/kg/17W- | LIVER: “HEPATITIS | American Journal of |
| | | | (343 mg/kg) | (HEPATOCELLULAR | Gastroenterology. |
| | | | | NECROSIS), DIFFUSE” | Vol. 91, Pg. 1641, |
| | | | | LIVER: LIVER | 1996. |
| | | | | FUNCTION TESTS | |
| | | | | IMPAIRED | |
| | | | | BLOOD: EOSINOPHILIA | |
| Mouse | LD50 | intracerebral | 38 mg/kg | BEHAVIORAL: | Chemotherapy Vol. |
| | | | (38 mg/kg) | “HALLUCINATIONS, | 26, Pg. 196, 1980. |
| | | | | DISTORTED | |
| | | | | PERCEPTIONS” | |
| | | | | BEHAVIORAL: | |
| | | | | EXCITEMENT | |
| | | | | MUSCULOSKELETAL: | |
| | | | | CHANGES IN TEETH | |
| | | | | AND SUPPORTING | |
| | | | | STRUCTURES | |
| Mouse | LD50 | intraperitoneal | 310 mg/kg | | “Antibiotics: Origin, |
| | | | (310 mg/kg) | | Nature, and |
| | | | | | Properties,” |
| | | | | | Korzyoski, T., et al., |
| | | | | | eds., Washington, |
| | | | | | DC, American Soc. |
| | | | | | for Microbiology, |
| | | | | | 1978 Vol. 1, Pg. 501, |
| | | | | | 1978. |
| Mouse | LD50 | intravenous | 140 mg/kg | | “Antibiotics: Origin. |
| | | | (140 mg/kg) | | Nature, and |
| | | | | | Properties,” |
| | | | | | Korzyoski, T., et al., |
| | | | | | eds., Washington, |
| | | | | | DC, American Soc. |
| | | | | | for Microbiology, |
| | | | | | 1978 Vol. 1, Pg. 501, |
| | | | | | 1978. |
| Mouse | LD50 | Oral | 3100 mg/kg | | “Antibiotics: Origin, |
| | | | (3100 mg/kg) | | Nature, and |
| | | | | | Properties,” |
| | | | | | Korzyoski, T., et al., |
| | | | | | eds., Washington, |
| | | | | | DC, American Soc. |
| | | | | | for Microbiology, |
| | | | | | 1978 Vol. 1, Pg. 501, |
| | | | | | 1978. |
| Women | TDLo | Oral | 8 mg/kg | SKIN AND | American Journal of |
| | | | (8 mg/kg) | APPENDAGES (SKIN): | Medicine. Vol. 109, |
| | | | | “DERMATITIS, | Pg. 340, 2000. |
| | | | | ALLERGIC: AFTER | |
| | | | | SYSTEMIC EXPOSURE” | |
| Women | TDLo | Oral | 28 mg/kg/2W-I | BRAIN AND | Annals of Internal |
| | | | (28 mg/kg) | COVERINGS: | Medicine. Vol. 127, |
| | | | | “CHANGES IN | Pg. 168, 1997. |
| | | | | CIRCULATION | |
| | | | | (HEMORRHAGE, | |
| | | | | THROMBOSIS, ETC.)” | |
| | | | | BEHAVIORAL: | |
| | | | | HEADACHE | |
| | | | | SENSE ORGANS AND | |
| | | | | SPECIAL SENSES: | |
| | | | | VISUAL FIELD | |
| | | | | CHANGES: EYE | |
| Women | TDLo | oral | 100 mg/kg | KIDNEY, URETER, AND | British Medical |
| | | | (100 mg/kg) | BLADDER: | Journal. Vol. 1, Pg. |
| | | | | INTERSTITIAL | 524, 1979. |
| | | | | NEPHRITIS | |
| | | | | KIDNEY, URETER, AND | |
| | | | | BLADDER: | |
| | | | | HEMATURIA | |
| | | | | KIDNEY, URETER, AND | |
| | | | | BLADDER: | |
| | | | | PROTEINURIS | |
| Women | TDLo | Oral | 112 mg/kg/4W-I | LIVER: LIVER | American Journal of |
| | | | (112 mg/kg) | FUNCTION TESTS | Gastroenterology. |
| | | | | IMPAIRED | Vol. 91, Pg. 1641, |
| | | | | SKIN AND | 1996. |
| | | | | APPENDAGES (SKIN): | |
| | | | | “DERMATITIS, OTHER: | |
| | | | | AFTER SYSTEMIC | |
| | | | | EXPOSURE” | |
| Women | TDLo | Oral | 730 mg/kg/1Y-I | BEHAVIORAL: | American Journal of |
| | | | (730 mg/kg) | ANOREXIA (HUMAN | Gastroenterology. |
| | | | | GASTROINTESTINAL: | Vol. 91, Pg. 1641, |
| | | | | NAUSEA OR VOMITING | 1996. |
| | | | | LIVER: LIVER | |
| | | | | FUNCTION TESTS | |
| | | | | IMPAIRED | |
| Women | TDLo | Oral | 1204 mg/kg/86W | MUSCULOSKELETAL: | British Journal of |
| | | | (1204 mg/kg) | JOINTS | Rheumatology. Vol. |
| | | | | | 33, Pg. 674, 1994. |
| Women | TDLo | oral | 17520 mg/kg/12 | SENSE ORGANS AND | American Journal of |
| | | | (17520 mg/kg) | SPECIAL SENSES: | Ophthalmology. |
| | | | | OTHER: EYE | Vol. 125, Pg. 396, |
| | | | | MUSCULOSKELETAL: | 1998. |
| | | | | CHANGES IN TEETH | |
| | | | | AND SUPPORTING | |
| | | | | STRUCTURES | |
| | | | | SKIN AND | |
| | | | | APPENDAGES (SKIN): | |
| | | | | “DERMATITIS OTHER: | |
| | | | | AFTER SYSTEMIC | |
| | | | | EXPOSURE” | |

We prefer the 7-Dimethylamino-6-demethyl-6-deoxytetracycline be formulated as an oral formulation. We prefer the oral formulation provide in vivo release of the 7-Dimethylamino-6-demethyl-6-deoxytetracycline over an extended period of time (as opposed to an oral formulation which provides immediate release into the patient's body). For example, the formulation may dissolve at a rate which releases the 7-Dimethylamino-6-demethyl-6-deoxytetracycline at a rate of not more than about 40 percent of the total after fifteen minutes, from about 50 to 80 percent after thirty minutes, at least 70 percent after forty-five minutes, and 100 percent after sixty minutes.

We prefer to provide this by providing the 7-Dimethylamino-6-demethyl-6-deoxytetracycline in a slowly dissolving dosage form. One example of a slowly dissolving dosage form is controlled-release pellets in a gelatin capsule. One approach to doing this is taught by Joseph J. VALOROSE, Jr., et al., Novel Controlled Release Formulations . . . , U.S. Pat. No. 4,837,030 (6 Jun. 1989). Other techniques of preparing controlled-release formulations are known in the art.

### Skin Cleanser

We prefer the skin cleanser to be a gentle, non-soap formulation to avoid drying the skin. We prefer this formula to be made of a base of water and Aloe barbadensis leaf juice. To this, we prefer to add glycerin, sodium PCA, panthenol, phospholipids, ascorbyl palmitate, tocopheryl acetate, retinyl palmitate, chondroitin sulfate, sodium hyaluronate, Octoxynol-9™, ethoxydiglycol, sodium benzoate, imidazolidinyl urea and disodium EDTA.

Alternatively, one can provide a cleanser made of a base of water and sodium laureth sulfate. To this base, we add cocamidopropyl betaine, cocamide MEA, polyquaternium-7, PEG-12, dimethicone, disodium cocamphodiacetate, panthenol, PEG-150 distearate, coenzyme Q-10 (ubiquinone), phenoxyethanol, sodium chloride, methylparaben, propylparaben, citric acid and disodium EDTA. This provides a foaming cleanser which gently cleans the skin.

We prefer the skin cleanser to include components to sooth the skin. For example, we prefer to include green tea (Camellia sinensis) extract.

The cleanser may be buffered to an appropriate pH to minimize the likelihood of skin irritation. We prefer that the cleanser have no added perfumes, to minimize the possibility that the cleanser will exacerbate dermal irritation.

We prefer to provide the cleanser in the form of a pre-moistened towel or wipe. Alternatively, it may be provided as a gel, bar, et cetera.

### Skin Moisturizer

After the patient uses the skin cleanser, we prefer the patient to then use a skin moisturizer which is light, non-greasy and soothing. We prefer to use a base made of purified water and bisabolol. To this, we prefer to include cosmetically-attractive botanicals such as cucumis sativus (cucumber) fruit extract, silybum marianum fruit extract, chamomilla recutita (matricaria) flower extract or camellia sinensis leaf extract. We also prefer to refine the attractiveness of the topical formulation by including sodium hyaluronate, carbomer, triethanolamine, diazolidinyl urea, methylparaben and tetrasodium edta.

Alternatively, one may provide a moisturizing base made of water and cetearyl alcohol. With this, we prefer to include PPG-2 myristyl ether propionate, squalane, dimethicone, polysorbate-60, polysorbate-20, hydroxycellulose, carbomer, butylene glycol, laureth-3, ethylene brassylate, beeswax, triethanolamine, methylparaben, propylparaben, imidazolidinyl urea, benzyl alcohol and disodium EDTA.

We prefer to include components which sooth skin irritation; these include Aloe barbadensis leaf juice (aloe vera gel), glycerine, green tea (Camellia sinensis) extract, acetyl dipeptide-1 cetyl ester and bisabolol.

To augment skin soothing, we prefer to include in our kit a dermal masque preparation. The composition of masque preparations are known in the art. For example, we prefer a masque base of algae extract in aqueous glycerin. We prefer to include soothing and anti-inflammatory botanicals such as haslea ostrearia (blue algae) extract, palmaria palmata (sea parsley) extract, sea whip extract, macrocystis pyrifera (kelp) extract, camellia sinensis leaf extract. We also prefer to include dimethicone, caprylic/capric triglyceride, xanthan gum, cyclopentasiloxane, hydrolyzed wheat protein, carbomer, PVP, sodium polyacrylate, trideceth-6, PEG/PPG 18/18 dimethicone, chlorophyllin-copper complex, DNA, caprylyl glycol, phenoxyethanol, sorbic acid and disodium EDTA to make a cosmetically-elegant formulation.

### Packaging

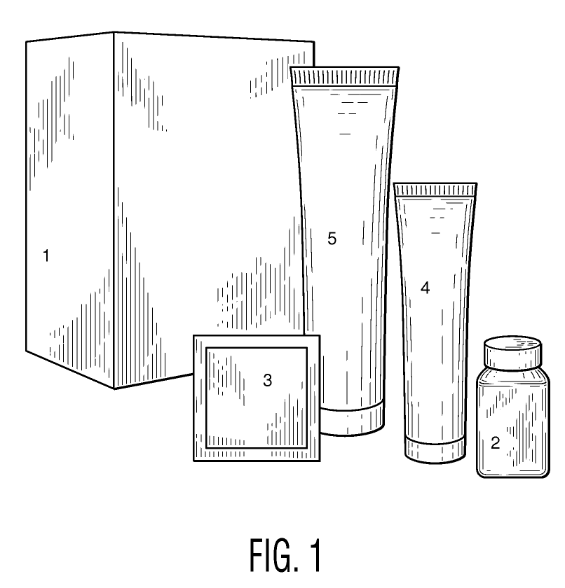

We prefer the various components to be packed together in a box. We show this in FIG. 1, showing [1] a box containing [2] a bottle or vial of controlled-release 7-Dimethylamino-6-demethyl-6-deoxytetracycline capsules; [3] an envelope containing a pre-moistened towel saturated with a dermatologically-acceptable skin cleanser; [4] a tube of a dermatologically-acceptable skin moisturizer, and [5] a tube containing a dermatologically-acceptable skin masque.

Other suitable packaging may, of course, be used. For example, one could provide a shrink-wrapped collection of three jars; one for each of 7-Dimethylamino-6-demethyl-6-deoxytetracycline, moisturizer and cleanser. Alternatively, one could provide the three aforementioned components in tubes, and provide the various tubes in a plastic or metal display rack. One of skill in the art may readily design attractive alternatives; we thus use the term “packaging” in our claims to encompass everything which is included in the Federal Food, Drug & Cosmetic Act definition of “labeling.”

We claim:

1. A kit for the treatment of acne vulgaris, the kit comprising:
a. 7-Dimethylamino-6-demethyl-6-deoxytetracycline,
i. said 7-Dimethylamino-6-demethyl-6-deoxytetracycline in a formulation acceptable for oral administration,
ii. said 7-Dimethylamino-6-demethyl-6-deoxytetracycline presented in an amount effective for treatment of acne vulgaris when administered orally; and
iii. said 7-Dimethylamino-6-demethyl-6-deoxytetracycline formulated to provide controlled release of 7-Dimethylamino-6-demethyl-6-deoxytetracycline in vivo;
b. a non-soap skin cleanser having a formulation which avoids exacerbating acne-vulgaris associated skin irritation; said non-soap skin cleanser further having a formulation which avoids exacerbating 7-Dimethylamino-6-demethyl-6-deoxytetracycline associated skin irritation; and
c. a non-greasy skin moisturizer having a formulation which avoids exacerbating acne-vulgaris associated skin irritation; said non-greasy skin moisturizer further having a formulation which avoids exacerbating 7-Dimethylamino-6-demethyl-6-deoxytetracycline associated skin irritation.

2. The kit of claim 1, said amount of 7-Dimethylamino-6-demethyl-6-deoxytetracycline being a dosage of from about 50 milligrams to about 200 milligrams of 7-Dimethylamino-6-demethyl-6-deoxytetracycline per dose.

3. The kit of claim 1, wherein said non-soap skin cleanser includes Aloe barbadensis extract.

4. The kit of claim 1, wherein said non-greasy skin moisturizer includes a topical anti-irritant component.

5. The kit of claim 4, said topical anti-irritant component selected from the group consisting of: Aloe barbadensis leaf juice (aloe vera gel); glycerin, green tea (Camellia sinensis) extract; acetyl dipeptide-1 cetyl ester; and bisabolol.

6. The kit of claim 5, said topical anti-irritant component comprising Aloe barbadensis leaf juice (aloe vera gel), glycerine, green tea (Camellia sinensis) extract, acetyl dipeptide-1 cetyl ester and bisabolol.

7. The kit of claim 1, wherein
a. said amount of 7-Dimethylamino-6-demethyl-6-deoxytetracycline being a dosage of from about 50 milligrams to about 200 milligrams of 7-Dimethylamino-6-demethyl-6-deoxytetracycline per dose; and
b. said non-soap skin cleanser includes Aloe barbadensis extract; and
c. said non-greasy skin moisturizer includes a topical anti-irritant component selected from the group consisting of: Aloe barbadensis leaf juice (aloe vera gel); glycerine, green tea (Camellia sinensis) extract; acetyl dipeptide-1 cetyl ester; and bisabolol.

8. A method for the treatment of acne vulgaris, the method comprising:
a. providing to a patient in need thereof an effective amount of the 7-Dimethylamino-6-demethyl-6-deoxytetracycline of claim 1; and
b. providing to said patient the non-soap skin cleanser of claim 1; and
C. providing to said patient the non-greasy skin moisturizer of claim 1.

9. The method of claim 8, said amount of 7-Dimethylamino-6-demethyl-6-deoxytetracycline being a dosage of from about 50 milligrams to about 200 milligrams of 7-Dimethylamino-6-demethyl-6-deoxytetracycline per dose.

10. The method of claim 8, wherein said non-soap skin cleanser includes Aloe barbadensis extract.

11. The method of claim 8, wherein said non-greasy skin moisturizer includes a topical anti-irritant component.

12. The method of claim 11, said topical anti-irritant component selected from the group consisting of: Aloe barbadensis leaf juice (aloe vera gel); glycerin, green tea (Camellia sinensis) extract; acetyl dipeptide-1 cetyl ester; and bisabolol.

13. The method of claim 12, said topical anti-irritant component comprising Aloe barbadensis leaf juice (aloe vera gel), glycerine, green tea (Camellia sinensis) extract, acetyl dipeptide-1 cetyl ester and bisabolol.

14. The method of claim 8, wherein
a. said amount of 7-Dimethylamino-6-demethyl-6-deoxytetracycline being a dosage of from about 50 milligrams to about 200 milligrams of 7-Dimethylamino-6-demethyl-6-deoxytetracycline per dose; and
b. said non-soap skin cleanser includes Aloe barbadensis extract; and
c. said non-greasy skin moisturizer includes a topical anti-irritant component selected from the group consisting of: Aloe barbadensis leaf juice (aloe vera gel); glycerine, green tea (Camellia sinensis) extract; acetyl dipeptide-1 cetyl ester; and bisabolol.
